(19) United States
(12) Patent Application Publication
(10) Pub. No.: US 2019/0223905 A1
(43) Pub. Date: Jul. 25, 2019

(54) VISUALLY ASSISTED SINGLE-SITE PORT CLOSURE GUIDE AND METHODS OF USE

(71) Applicant: Zach Zeller, San Antonio, TX (US)

(72) Inventor: Zach Zeller, San Antonio, TX (US)

(21) Appl. No.: 16/255,788

(22) Filed: Jan. 23, 2019

Related U.S. Application Data
(60) Provisional application No. 62/620,655, filed on Jan. 23, 2018.

(51) Int. Cl. A61B 17/34; A61B 17/02

(52) U.S. Cl. CPC A61B 17/3423; A61B 2017/3445; A61B 17/0218

(57) ABSTRACT

Disclosed herein is a device and method of use for closure guides. More specifically, the present invention generally relates to a device and method of use for a visually assisted single-site port closure guide. In an embodiment the closure guide device is comprised of a main body element having a cone portion, a bulb portion, a center vertical cannula portion, and two angled channel portions. In other embodiments, the main body element is further comprised of a cap portion. In yet other embodiments, the main body element is further comprised of left and right wing portions.

### CROSS-REFERENCE TO RELATED APPLICATIONS

This application claims the benefit under Title 35 United States Code § 119(e) of U.S. Provisional Patent Application Ser. No. 62/620,655; Filed: Jan. 23, 2018, the full disclosure of which is incorporated herein by reference.

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### INCORPORATING-BY-REFERENCE OF MATERIAL SUBMITTED ON A COMPACT DISC

Not applicable

### SEQUENCE LISTING

Not applicable

### FIELD OF THE INVENTION

The present invention generally relates to a device and method of use for closure guides. More specifically, the present invention generally relates to a device and method of use for a visually assisted single-site port closure guide.

### BACKGROUND OF THE INVENTION

Without limiting the scope of the disclosed device, the background is described in connection with a novel device and method of use for a visually assisted single-site port closure guide.

Today many surgeries are performed within a patient's body with the assistance of elongated tools and cameras. The patient's abdomen is inflated and must stay inflated to allow access and visualization inside the abdominal cavity. Small five to fifteen millimeter defects are created around the cavity to allow access inside of the patient's body. In order to maintain inflation of the cavity or pneumo as well as accessibility, these sites must have a trocar placed inside the defect. A trocar is a direct access port with a hollow lumen and pressurized seals to maintain air pressure. They are purely designed to keep the defect open and to provide direct access. Trocars to not provide any role with the closing of the defect.

When a procedure is completed, the trocar is pulled out of the defect and a closure device is placed inside the existing defect. When the trocar is placed inside of the abdomen, all layers of the abdominal wall were punctured. If a defect is ten millimeters or larger as a rule of thumb, the fascial layer at the bottom must be sutured to in order to prevent herniation through the port sites following the procedure. To close these defects, surgeons use laparoscopic port site closure devices. It is possible to hand suture but the defect is extremely small and the fascial layer is near the deepest point which makes it extremely difficult to be accurate. Closing a port site involves using a suture passer, a sharp needle-like post with jaws at the end to hold a suture, inside the patient to pass suture from one side of the defect to the other in order to bring the edges of the fascial layer together. Having a sharp inside of the patient's body requires optimal pneumo and direct camera visualization. Current methods and approaches involve placing a camera in an adjacent port to allow visualization of the closure.

While all of the aforementioned devices and approaches may fulfill their unique purposes, none of them fulfill the need for a practical, effective, and efficient means for a closure guide.

Therefore, the present invention proposes a novel device and method of use for a visually assisted single-site port closure guide to address the shortcomings in the prior art.

### BRIEF SUMMARY OF THE INVENTION

The present invention, therefore, provides for a closure guides. More specifically, the present invention generally relates to a device and method of use for a visually assisted single-site port closure guide.

In an embodiment, the closure guide device is comprised of a main body element having a cone portion, a bulb portion, a center vertical cannula portion, and two angled channel portions. The cone portion is cylindrical in shape. In embodiments, the cone portion is tapered from top to bottom. The bulb portion is positioned at the tip or end of the cone portion and is clear. In embodiments, the bulb portion is made of a clear polymer or glass. The center vertical cannula portion is positioned through the longitudinal axis of the of the cone portion being open at the top and terminating at the bottom of the cone portion at the bulb portion. The two angled channel portions are for receiving and passing through a suture passer or sharp. In other embodiments, the main body element is further comprised of a cap portion. In yet other embodiments, the main body element is further comprised of left and right wing portions.

In summary, the present invention discloses a novel device and method of use for a visually assisted single-site port closure guide.

### BRIEF DESCRIPTION OF THE SEVERAL VIEWS OF THE DRAWINGS

For a more complete understanding of the features and advantages of the present invention, reference is now made to the detailed description of the invention along with the accompanying figures in which:

FIG. 1 is a front perspective view of the closure guide in accordance with embodiments of the disclosure;

FIG. 2 is a front view of the closure guide in accordance with embodiments of the disclosure;

FIG. 3 is a back view of the closure guide in accordance with embodiments of the disclosure;

FIG. 4 is a left side view of the closure guide in accordance with embodiments of the disclosure;

FIG. 5 is a right side view of the closure guide in accordance with embodiments of the disclosure;

FIG. 6 is a bottom view of the closure guide in accordance with embodiments of the disclosure;

FIG. 7 is a top view of the closure guide in accordance with embodiments of the disclosure;

FIG. 8 is a front view of a camera utilized with the closure guide in accordance with embodiments of the disclosure;

FIG. 9 is a front perspective view of a camera utilized with the closure guide in accordance with embodiments of the disclosure;

FIG. 10 is a front view of the camera inserted through the closure guide in accordance with embodiments of the disclosure; and

FIG. 11 is a front view of a camera inserted through the closure guide and a suture passer inserted through the closure guide in accordance with embodiments of the disclosure.

### DETAILED DESCRIPTION OF THE INVENTION

Disclosed herein is an improved device and method of use directed to a visually assisted single-site port closure guide. The numerous innovative teachings of the present invention will be described with particular reference to several embodiments (by way of example, and not of limitation).

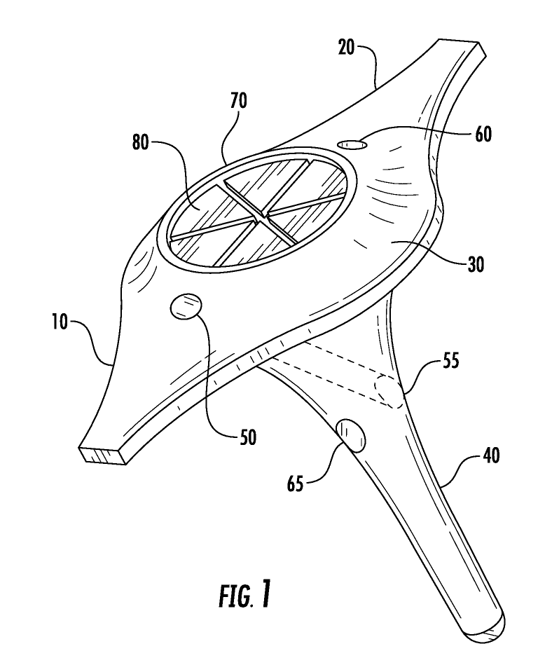

Reference is first made to FIG. 1, a front perspective view of the closure guide in accordance with embodiments of the disclosure. The device is a single closure guide that is placed inside the defect once the trocar is removed. Illustrated here are some of the portions of the closure guide such as the cone portion 40, the two angled channel portions 50, 55, 60, 65, the cap portion 30, the left wing portion 10, the right wing portion 20, the center vertical cannula portion 70, and the center vertical cannula portion cover 80.

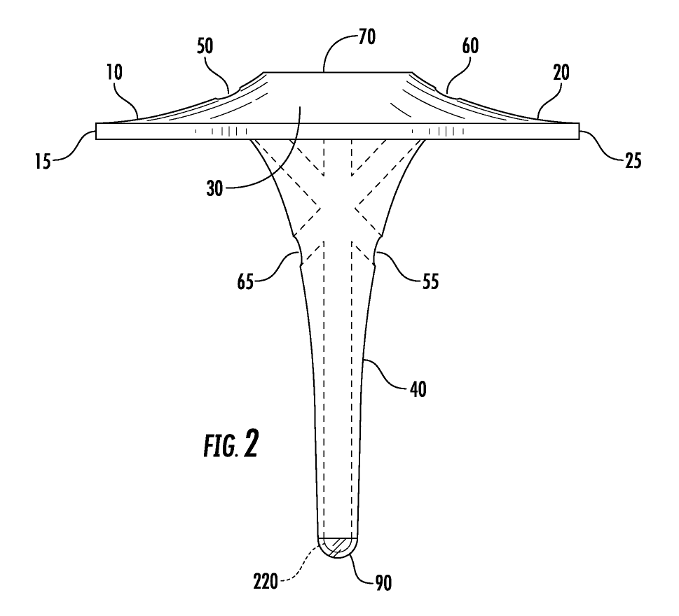

Reference is next made to FIG. 2, a front view of the closure guide in accordance with embodiments of the disclosure. In an embodiment, the closure guide device is comprised of a main body element having a cone portion 40, a bulb portion 90, a center vertical cannula portion 70, and two angled channel portions 50, 55, 60, 65. The cone portion 40 is cylindrical in shape. In embodiments, the cone portion 40 is tapered from top to bottom. In further embodiments the cone portion 40 is tapered from five millimeters at the bottom tip to twelve millimeters at the top. The gradual increase in diameter assists in maintaining pneumo inside of the patient and prevents any additional trauma to the defect during closure. The bulb portion 90 is positioned at the tip or end of the cone portion 40 and is clear. In embodiments, the bulb portion 90 is made of a clear polymer or glass. The bulb portion 90 in embodiments may be removed allowing the tip or end of the cone portion 40 to be open. This allows a captured or non-captured configuration. The center vertical cannula portion 70 is positioned through the longitudinal axis of the of the cone portion 40 being open at the top and terminating at the bottom of the cone portion 40 at the bulb portion 90. In embodiments, the center vertical cannula portion 70 is five millimeters. The two angled channel portions 50, 55, 60, 65 are for receiving and passing through a suture passer or sharp through opposing sides of the defect.

In other embodiments, the main body element is further comprised of a cap portion 30 for evenly distribute the weight across the top of the defect, maintain pressure, and prevent the device from slipping inside of the patient. In embodiments, the cap portion 30 is twenty four millimeters.

In yet other embodiments, the main body element is further comprised of a left wing portion 10 and a right wing portion 20. The left wing portion 10 has a left end 15 and the right wing portion 20 has a right end 25.

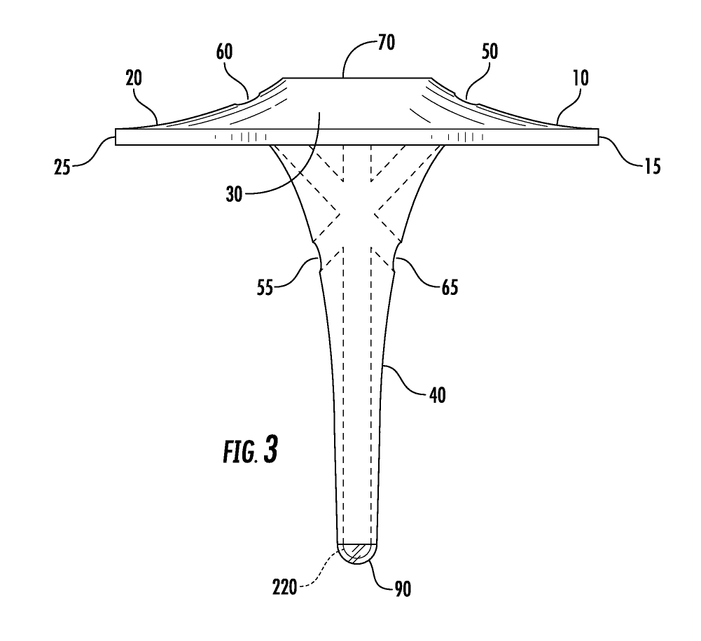

Reference is now made to FIG. 3, a back view of the closure guide in accordance with embodiments of the disclosure. In this illustration is shown the back side and the two angled channel portions 50, 55, 60, 65.

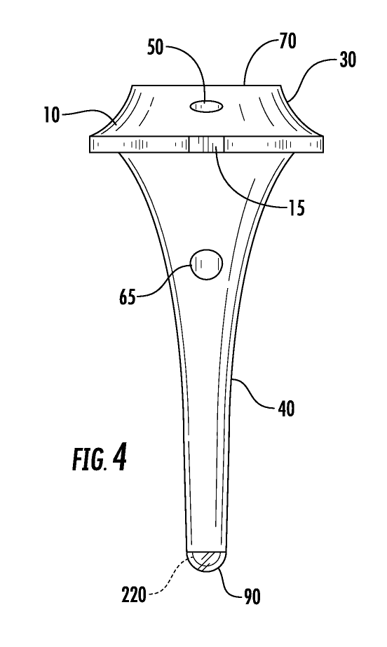

Reference is next made to FIG. 4, a left side view of the closure guide in accordance with embodiments of the disclosure.

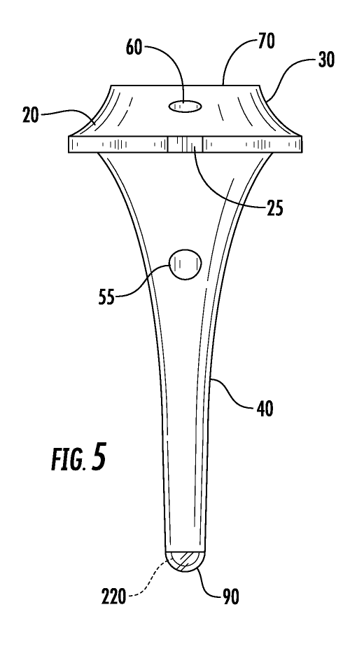

Reference is now made to FIG. 5, a right side view of the closure guide in accordance with embodiments of the disclosure.

Reference is next made to FIG. 6, a bottom view of the closure guide in accordance with embodiments of the disclosure. In this view is illustrated the bottom side of the cap portion 35, the bottom side of the left wing portion 18, and the bottom side of the right wing portion 28.

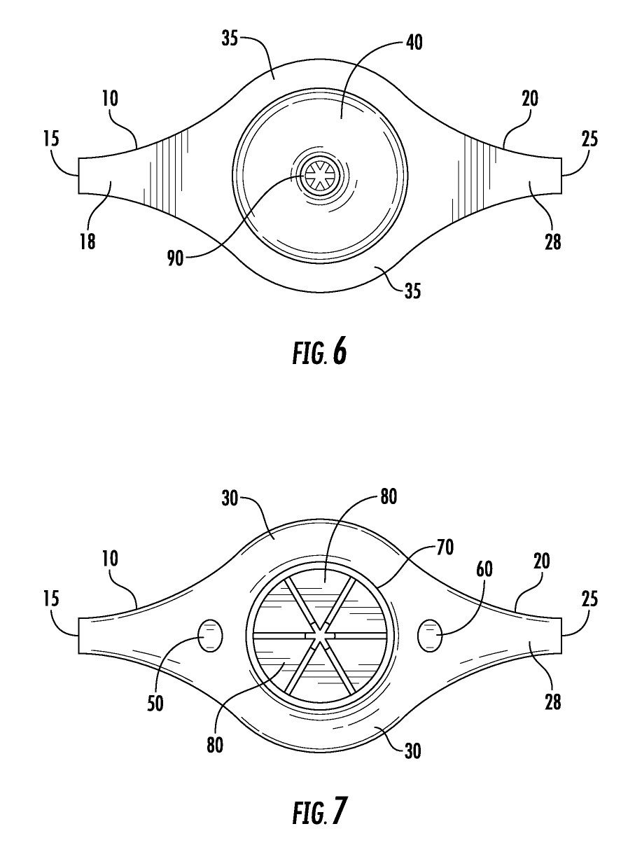

Reference is now made to FIG. 7, a top view of the closure guide in accordance with embodiments of the disclosure. Illustrated in this figure is the center vertical cannula portion 70 and the center vertical cannula portion cover 80.

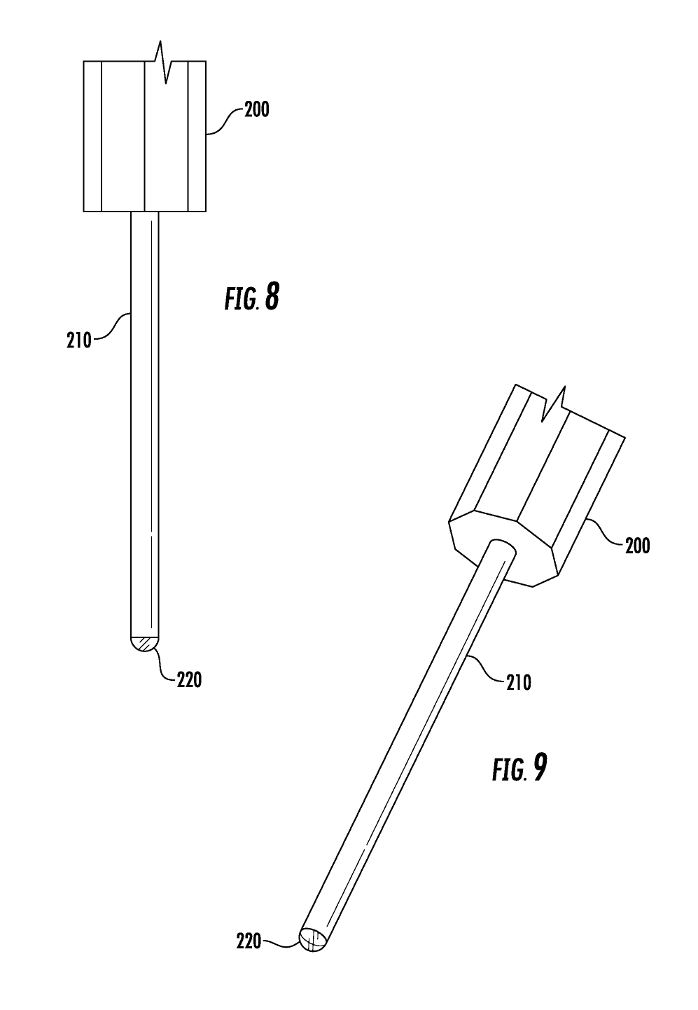

Reference is next made to FIG. 8, a front view of a camera utilized with the closure guide in accordance with embodiments of the disclosure. Illustrated in this figure is the camera grip 200, the camera probe 210, and the camera tip 220.

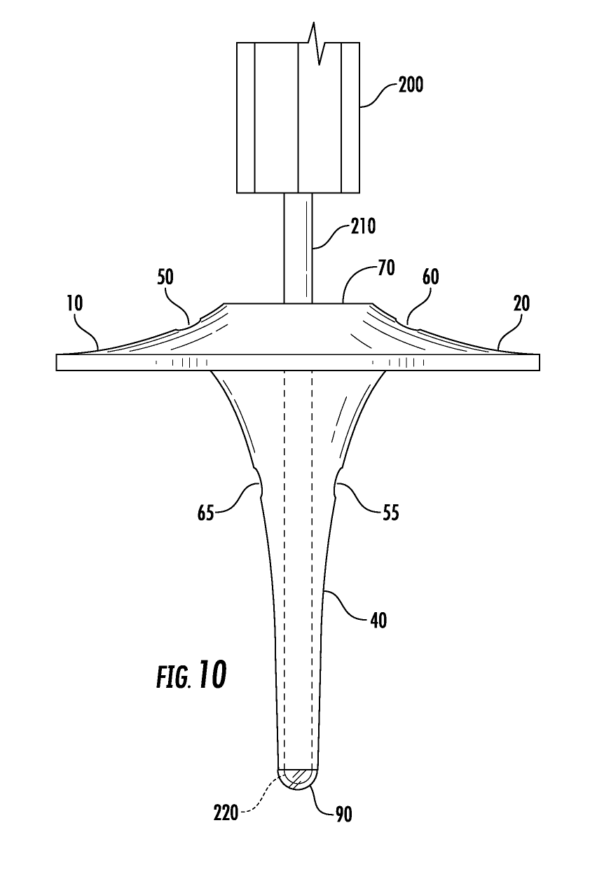

Reference is now made to FIG. 9, a front perspective view of a camera utilized with the closure guide in accordance with embodiments of the disclosure. Illustrated in this perspective figure is the camera grip 200, the camera probe 210, and the camera tip 220.

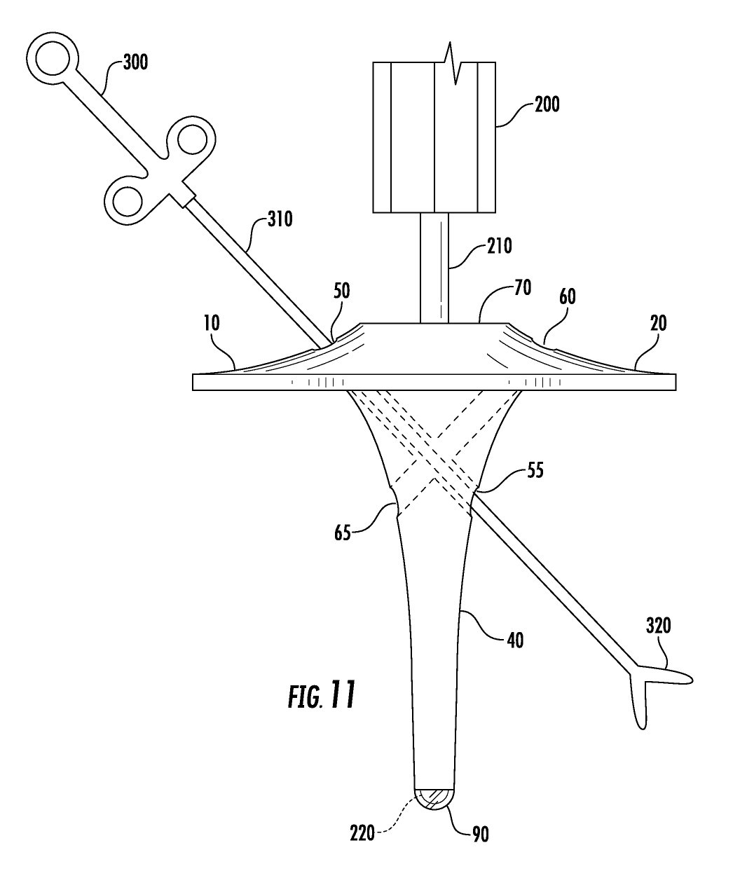

Reference is next made to FIG. 10, a front view of the camera inserted through the closure guide in accordance with embodiments of the disclosure. Illustrated in this figure is the first step where the camera probe 210 is inserted through the closure guide's center vertical cannula portion 70 until the camera tip 220 is flush or reaches the end and presses against the bulb portion 90. This is to allow visualization.

Reference is lastly made to FIG. 11, a front view of a camera inserted through the closure guide and a suture passer inserted through the closure guide in accordance with embodiments of the disclosure. Illustrated in this figure is the second step where a single suture is loaded in to a suture passer and passed through one of the angled channel portion 50, 55 and left inside, directly under the camera lens, of the patient temporarily. Illustrated in this figure is the suture passer handle 300, the suture passer sharp needle-like post portion 310, and suture passer jaws 320. The suture passer is removed and is now inserted into the opposite angled channel portion 60, 65. The suture is grasped from the opposite channel and pulled through to provide a single suture crossing both sides of the defect. The surgeon will then have the guide with a suture passed from one side to the other. The surgeon is able to visualize all of the closure upon finalization of the case. The device is removed and the suture is tied. This device is designed to close single port sites where no other port sites are available to hot a camera.

In brief, the invention is directed to a device and method of use for a closure guide.

The disclosed device and method of use is generally described, with examples incorporated as particular embodiments of the invention and to demonstrate the practice and advantages thereof. It is understood that the examples are given by way of illustration and are not intended to limit the specification or the claims in any manner.

To facilitate the understanding of this invention, a number of terms may be defined below. Terms defined herein have meanings as commonly understood by a person of ordinary skill in the areas relevant to the present invention. Terms such as “a”, “an”, and “the” are not intended to refer to only a singular entity, but include the general class of which a specific example may be used for illustration. The terminology herein is used to describe specific embodiments of the invention, but their usage does not delimit the disclosed device or method of use, except as may be outlined in the claims.

Alternative applications for this invention include using this device or method of use in any application where a visually assisted closure guide is needed. Consequently, any embodiments comprising a one piece or multi piece device having the structures as herein disclosed with similar function shall fall into the coverage of claims of the present invention and shall lack the novelty and inventive step criteria.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific device and method of use described herein. Such equivalents are considered to be within the scope of this invention and are covered by the claims.

All publications, references, patents, and patent applications mentioned in the specification are indicative of the level of those skilled in the art to which this invention pertains. All publications, references, patents, and patent applications are herein incorporated by reference to the same extent as if each individual publication, reference, patent, or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

The device and/or methods of use disclosed and claimed herein can be made and executed without undue experimentation in light of the present disclosure. While the device and methods of use of this invention have been described in terms of preferred embodiments, it will be apparent to those skilled in the art that variations may be applied to the device and/or methods of use and in the steps or in the sequence of steps of the method of use described herein without departing from the concept, spirit, and scope of the invention.

More specifically, it will be apparent that certain components, which are both shape and material related, may be substituted for the components described herein while the same or similar results would be achieved. All such similar substitutes and modifications apparent to those skilled in the art are deemed to be within the spirit, scope, and concept of the invention as defined by the appended claims.

What is claimed is:

1. A closure guide device comprised of:
a main body element having a cone portion, a bulb portion, a center vertical cannula portion, and two angled channel portions.

2. The closure device of claim 1, wherein the main body element is further comprised of a cap portion.

3. The closure device of claim 1, wherein the main body element is further comprised of left and right wing portions.

4. A method of use for a closure guide device as herein disclosed.
